(19) United States
(12) Patent Application Publication
(10) Pub. No.: US 2006/0252978 A1
(43) Pub. Date: Nov. 9, 2006

(54) Biofeedback eyewear system

(76) Inventors: Michael A. Vesely, Santa Cruz, CA (US); Nancy L. Clemens, Santa Cruz, CA (US)

Correspondence Address: Tue Nguyen, 496 Oliver Ave., Fremont, CA, 94539, US

(21) Appl. No.: 11/429,824

(22) Filed: May 8, 2006

Related U.S. Application Data
(60) Provisional application No. 60/679,631, filed on May 9, 2005.

(51) Int. Cl. A61M 21/00; A61B 5/04; A61B 5/00

(52) U.S. Cl. 600/27; 600/545; 600/549; 600/546

(57) ABSTRACT

A biofeedback eyewear system comprising stereo lenses, binaural audio and plurality of electrodes for biofeedback devices is disclosed. The stereo lenses comprise different left and right lenses such as 0° and 90° polarized lenses, allowing the user to view 3D images. The binaural audio comprises left and right headphones, allowing the user to hear 3D sound. The electrodes for biofeedback devices is preferably incorporated to the eyewear handles, bridge or frame, allowing the inputs of user's physical and mental status to a computer system. The disclosed biofeedback eyewear system forms a two-way communication between a user and a computer system.

This application claims priority from U.S. provisional applications Ser. No. 60/679,631, filed May 9, 2005, entitled “Biofeedback eyewear system”, which is incorporated herein by reference.

### FIELD OF INVENTION

The present invention relates generally to methods and apparatus for a two-way communication with a computer system, and more particularly, to a biofeedback eyewear system.

### BACKGROUND OF THE INVENTION

Stereo vision can be achieved due to different images received be the left and right eyes. Since the left and right eyes are normally separated by about 2 inches, the images of the same 3D structure received by these two eyes are slightly different. This difference is interpreted by the brain to create a 3D illusion, rendering depth of field to a 2D image.

Thus 3D display system simulates the actions of the two eyes to create the perception of depth, namely displaying a left image for the left eye and displaying a right image for the right eye with no interference between the two eyes. The methods to separate the left and right eyes and images include methods with glasses such as anaglyph method, special polarized glasses or shutter glasses, methods without using glasses such as a parallax stereogram, a lenticular method, and mirror method (concave and convex lens).

In anaglyph method, a display image for the right eye and a display image for the left eye are respectively superimpose-displayed in two colors, e.g., red and blue, and observation images for the right and left eyes are separated using color filters, thus allowing a viewer to recognize a stereoscopic image. The images are displayed using horizontal perspective technique with the viewer looking down at an angle. As with one eye horizontal perspective method, the eyepoint of the projected images has to be coincide with the eyepoint of the viewer, and therefore the viewer input device is essential in allowing the viewer to observe the three dimensional horizontal perspective illusions. From the early days of the anaglyph method, there are many improvements such as the spectrum of the red/blue glasses and display to generate much more realism and comfort to the viewers.

In polarized glasses method, the left eye image and the right eye image are separated by the use of mutually extinguishing polarizing filters such as orthogonally linear polarizer, circular polarizer, and elliptical polarizer. The images are normally projected onto screens with polarizing filters and the viewer is then provided with corresponding polarized glasses. The left and right eye images appear on the screen at the same time, but only the left eye polarized light is transmitted through the left eye lens of the eyeglasses and only the right eye polarized light is transmitted through the right eye lens.

Another way for stereoscopic display is the image sequential system. In such a system, the images are displayed sequentially between left eye and right eye images rather than superimposing them upon one another, and the viewer's lenses are synchronized with the screen display to allow the left eye to see only when the left image is displayed, and the right eye to see only when the right image is displayed. The shuttering of the glasses can be achieved by mechanical shuttering or with liquid crystal electronic shuttering. In shuttering glass method, display images for the right and left eyes are alternately displayed on a CRT in a time sharing manner, and observation images for the right and left eyes are separated using time sharing shutter glasses which are opened/closed in a time sharing manner in synchronism with the display images, thus allowing an observer to recognize a stereoscopic image.

Other way to display stereoscopic images is by optical method. In this method, display images for the right and left eyes, which are separately displayed on a viewer using optical means such as prisms, mirror, lens, and the like, are superimpose-displayed as observation images in front of an observer, thus allowing the observer to recognize a stereoscopic image. Large convex or concave lenses can also be used where two image projectors, projecting left eye and right eye images, are providing focus to the viewer's left and right eye respectively. A variation of the optical method is the lenticular method where the images form on cylindrical lens elements or two dimensional array of lens elements.

In addition to vision, audio and biofeedback are also critical components for a two-way communication between a computer system and a user.

### SUMMARY OF THE INVENTION

The present invention realizes that special glasses that prevent the interference between the two eyes are typically needed for the perception of 3D illusion, and thus discloses a biofeedback eyewear system comprising stereo lenses, binaural audio and electrodes for biofeedback devices.

The stereo lenses are preferably different left and right polarized lenses for light weight and ease of stereo display, but other stereo methods such as anaglyph, shutter glasses can also be used. The binaural audio is preferably different left and right headphones or earphones for perception of 3D sound, but monoaural audio can also be used.

The electrodes for biofeedback devices comprise sensor electrodes for brain wave measurement, blood pressure measurement, heart beat measurement, respiration measurement, perspiration measurement, skin conductance measurement, body temperature measurement, or muscle tension measurements, and preferably incorporated into the eyewear components such as the handles, the bridge, or the frame for light weight and ease of operation.

The biofeedback eyewear system permits a user to have two-way communication with a computer system.

### BRIEF DESCRIPTION OF THE DRAWINGS

FIG. 1 shows an embodiment of the present invention apparatus.

FIG. 2 shows the comparison of central perspective (Image A) and horizontal perspective (Image B).

FIG. 3 shows the horizontal perspective mapping of a 3D object onto the projection plane.

FIG. 4 shows the two-eye view of a stereo 3D display.

FIG. 5 shows an application of the present invention apparatus to brain balancing.

### DETAILED DESCRIPTION OF THE INVENTION

The present invention discloses a biofeedback eyewear system that uses parts of the eyewear such as the handle, the bridge, or the frame for an electrode for a biofeedback device. Since a biofeedback sensor electrode typically needs to contact the body for measuring the body response, and portions of the eyewear also contact the head, the eyewear system can combine the function of the eyeglasses together with the biofeedback.

The biofeedback device normally requires an electrode in contacting with the body. Typical electrodes are an electrode, or antenna, to receive the brain wave of the wearer. Other electrodes can be used to measure the skin conductance, the heart beat, the heart rate, the respiration, the perspiration, the stress level, the muscle tension. The electrode can be formed at the handle of the eyewear, or at the bridge where it contact the wearer's skin.

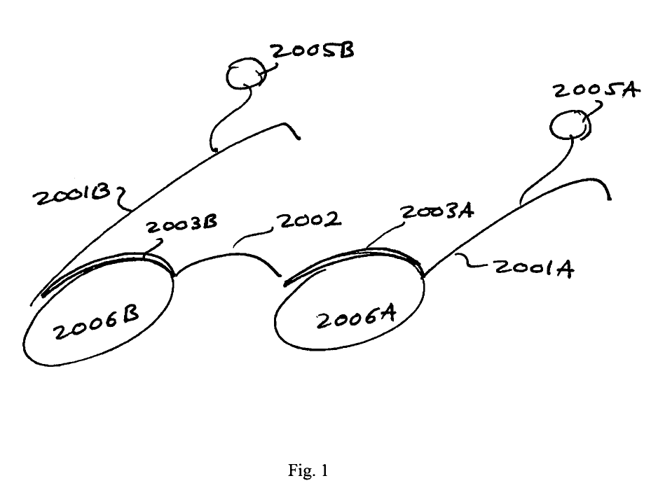

FIG. 1 shows an embodiment of the present invention biofeedback eyewear system. The biofeedback eyewear system has the shape of eyeglasses with two different lenses for the left eye 2006A and the right eye 2006B, and two earphones for the left ear 2005A and the right ear 2005B. Preferably, the lenses of the eyewear are configured to provide left and right separation, such as the left eye can only see the images designed for the left eye and not seeing the images designed for the right eye. For example, each of the lenses can comprise a 90° linearly separated polarizing lens, e.g. the left eye lens can be +90° or −90° linearly polarized with respect to the right eye lens. The left eye lens can be clockwise circular or elliptical polarized and the right eye lens can be counterclockwise circular or elliptical polarized. The lenses are preferably mounted on an eyewear frame, and connected by a bridge portion 2002. The bridge portion is typically configured to receive the nose of the wearer. The bridge normally contacts the top of the nose of the wearer, thus can be used as an electrode for a biofeedback device. The frame is also provided with a pair of generally rearwardly extending handles, a left handle 2001A and a right handle 2001B, configured to retain the eyewear. The handles normally contact the head of the wearer, thus can be used as a left electrode and a right electrode for a biofeedback device. The eyewear can comprise two lens frames for holding the lenses, a left frame 2003A for holding a left lens 2006A and a right frame 2003B for holding a right lens 2006B. The frames could contact the face of the wearer, and thus can be used as a left electrode and a right electrode for a biofeedback device. The handles, the frame and the bridge can be made from any conductive material for acting as an electrode, or an antenna for biofeedback devices.

The eyewear can further comprise a microphone, disposed anywhere on the frame, the bridge, or the handles. Optionally, the microphone and the earphones or headphones can be in the form of a bone conduction, in contact with the head, such that vibrations to and from the wearer can be travel through the bone. A speaker can act like a microphone, and thus two ways communication (microphone and speaker) can be achieved through a single speaker or microphone.

The audio devices and the electrodes are connected to a computer system to receive and provide the appropriate signals. The connection can be wired or preferably wireless. The eyewear audio portion is typically directed to the wearer through the use of transducers inside or covering the ear, such as earphones and headphones. Further, the audio device can include noise cancellation electronics to filter unwanted noise.

The eyewear is preferably configured to communicate via wireless protocols to the central computer system. With a wireless audio device, the eyewear system further comprises a power source, a transceiver and a signal antenna. The power source can be disposable or rechargeable batteries, or a solar panel.

The left and right lenses of the present invention biofeedback eyewear system are preferably applied to a horizontal perspective 3D display.

Horizontal perspective is a little-known perspective, sometimes called “free-standing anaglyph”, “phantogram”, or “projective anaglyph”. Normally, as in central perspective, the plane of vision, at right angle to the line of sight, is also the projected plane of the picture, and depth cues are used to give the illusion of depth to this flat image. In horizontal perspective, the plane of vision remains the same, but the projected image is not on this plane. It is on a plane angled to the plane of vision. Typically, the image would be on the ground level surface. This means the image will be physically in the third dimension relative to the plane of vision. Thus horizontal perspective can be called horizontal projection.

In horizontal perspective, the object is to separate the image from the paper, and fuse the image to the three dimension object that projects the horizontal perspective image. Thus the horizontal perspective image must be distorted so that the visual image fuses to form the free standing three dimensional figure. It is also essential the image is viewed from the correct eye points, otherwise the three dimensional illusion is lost. In contrast to central perspective images which have height and width, and project an illusion of depth, and therefore the objects are usually abruptly projected and the images appear to be in layers, the horizontal perspective images have actual depth and width, and illusion gives them height, and therefore there is usually a graduated shifting so the images appear to be continuous.

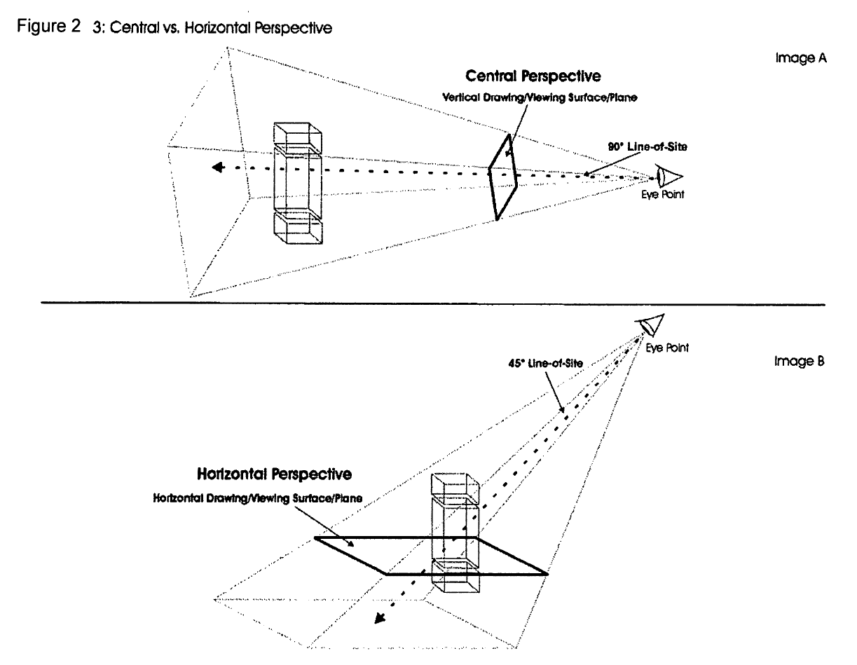

FIG. 2 compares key characteristics that differentiate central perspective and horizontal perspective. Image A shows key pertinent characteristics of central perspective, and Image B shows key pertinent characteristics of horizontal perspective.

In other words, in Image A, the real-life three dimension object (three blocks stacked slightly above each other) was drawn by the artist closing one eye, and viewing along a line of sight perpendicular to the vertical drawing plane. The resulting image, when viewed vertically, straight on, and through one eye, looks the same as the original image.

In Image B, the real-life three dimension object was drawn by the artist closing one eye, and viewing along a line of sight 45° to the horizontal drawing plane. The resulting image, when viewed horizontally, at 45° and through one eye, looks the same as the original image.

One major difference between central perspective showing in Image A and horizontal perspective showing in Image B is the location of the display plane with respect to the projected three dimensional image. In horizontal perspective of Image B, the display plane can be adjusted up and down, and therefore the projected image can be displayed in the open air above the display plane, i.e. a physical hand can touch (or more likely pass through) the illusion, or it can be displayed under the display plane, i.e. one cannot touch the illusion because the display plane physically blocks the hand. This is the nature of horizontal perspective, and as long as the camera eyepoint and the viewer eyepoint are at the same place, the illusion is present. In contrast, in central perspective of Image A, the three dimensional illusion is likely to be only inside the display plane, meaning one cannot touch it. To bring the three dimensional illusion outside of the display plane to allow viewer to touch it, the central perspective would need elaborate display scheme such as surround image projection and large volume.

One of the characteristics of horizontal perspective display is the projection onto the open space, and thus allowing a direct “touching” of the displayed images. Since the images are only projected images, there is no physical manifestation, and thus “touching” is not physically touching, but more like ghost touching, meaning the user can see by eyes and not feel by hands that the images are touched. The horizontal perspective images can also be displayed under the displayed surface, and thus a user cannot “touch” this portion. This portion can only be manipulated indirectly via a computer mouse or a joystick.

To synchronize the displayed images with the reality, the location of the display surface needs to be known to the computer. For a projection display, the projection screen is the display surface, but for a CRT computer monitor, the display surface is typically the phosphor layer, normally protected by a layer of glass. This difference will need to be taken into account to ensure accurate mapping of the images onto the physical world.

One element of horizontal perspective projection is the camera eyepoint, which is the focus of all the projection lines. The camera eyepoint is normally located at an arbitrary distance from the projection plane and the camera's line-of-sight is oriented at a 45° angle looking through the center. The user's eyepoint will need to be coinciding with the camera eyepoint to ensure minimum distortion and discomfort.

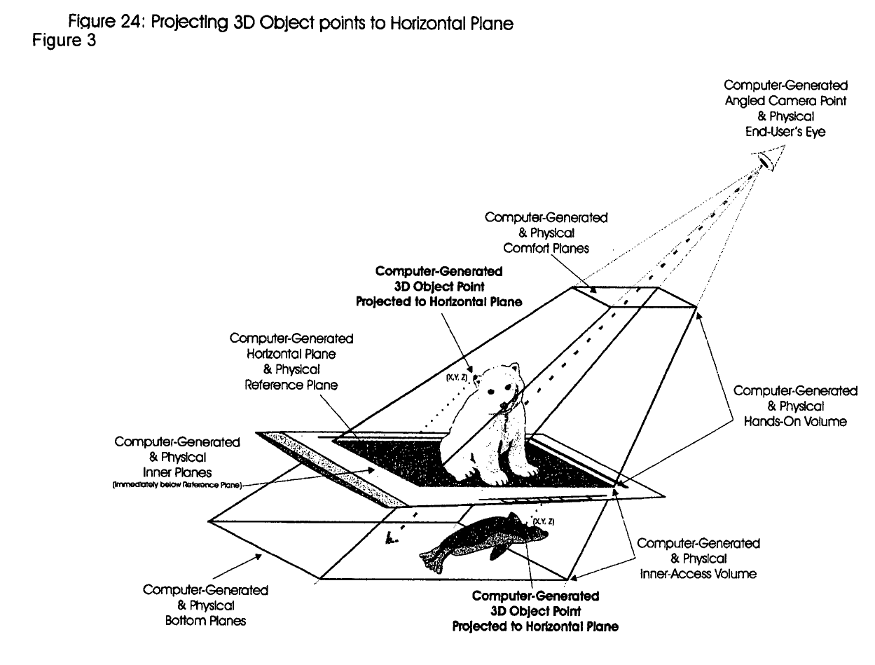

Mathematically, the projection lines to the camera eyepoint form a 45° pyramid. FIG. 3 illustrates this pyramid, which begins at the camera eyepoint and extending to the projection plane and beyond. The portion of the pyramid above the projection plane is a hands-on volume, where users can reach their hand in and physically “touch” a simulation. The portion of the pyramid under the projection plane is an inner-access volume, where users cannot directly interact with the simulation via their hand or hand-held tools. But objects in this volume can be interacted in the traditional sense with a computer mouse, joystick, or other similar computer peripheral.

The horizontal perspective display is preferably placed horizontally to the ground, meaning the projection plane must be at approximately a 45° angle to the end-user's line-of-sight for optimum viewing. Thus the CRT computer monitor is preferably positioned on the floor in a stand, so that the viewing surface is horizontal to the floor. This example use a CRT-type computer monitor, but it could be any type of viewing device, placed at approximately a 45° angle to the end-user's line-of-sight.

The system preferably displays stereoscopic images through stereoscopic 3D computer hardware to provide the user with multiple or separate left-and right-eye views of the same simulation. Thus stereoscopic 3D hardware devices include methods with glasses such as anaglyph method, special polarized glasses or shutter glasses, methods without using glasses such as a parallax stereogram, a lenticular method, and mirror method (concave and convex lens).

In anaglyph method, a display image for the right eye and a display image for the left eye are respectively superimpose-displayed in two colors, e.g., red and blue, and observation images for the right and left eyes are separated using color filters, thus allowing a viewer to recognize a stereoscopic image. In polarized glasses method, the left eye image and the right eye image are separated by the use of mutually extinguishing polarizing filters such as orthogonally linear polarizer, circular polarizer, and elliptical polarizer. Another way for stereoscopic display is the image sequential system. In such a system, the images are displayed sequentially between left eye and right eye images rather than superimposing them upon one another, and the viewer's lenses are synchronized with the screen display to allow the left eye to see only when the left image is displayed, and the right eye to see only when the right image is displayed. The shuttering of the glasses can be achieved by mechanical shuttering or with liquid crystal electronic shuttering. Other way to display stereoscopic images is by optical method. In this method, display images for the right and left eyes, which are separately displayed on a viewer using optical means such as prisms, mirror, lens, and the like, are superimpose-displayed as observation images in front of an observer, thus allowing the observer to recognize a stereoscopic image. Large convex or concave lenses can also be used where two image projectors, projecting left eye and right eye images, are providing focus to the viewer's left and right eye respectively. A variation of the optical method is the lenticular method where the images form on cylindrical lens elements or two dimensional array of lens elements.

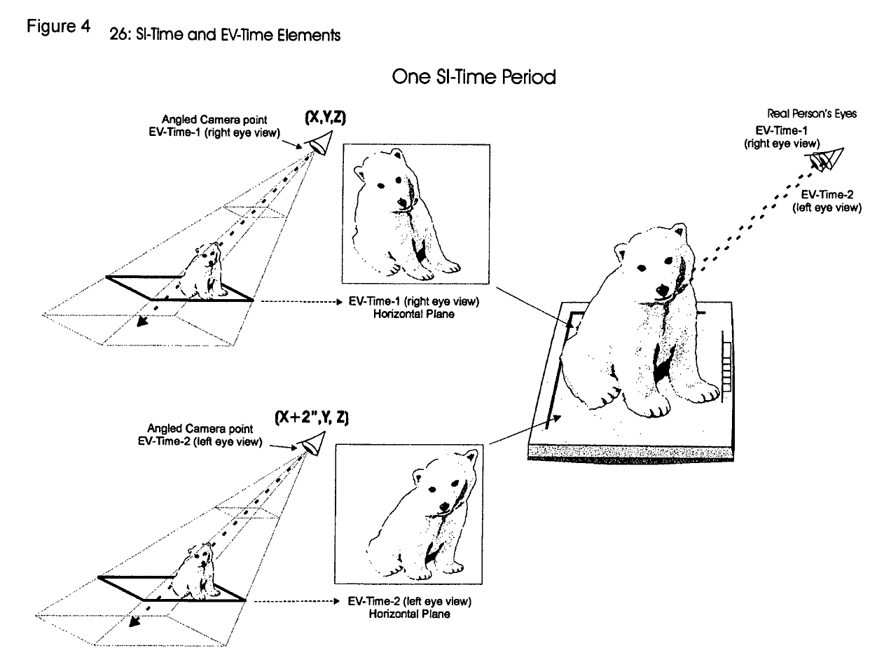

FIG. 4 illustrates the stereoscopic displayed images of the present invention horizontal perspective simulator. The user sees the bear cub from two separate vantage points, i.e. from both a right-eye view and a left-eye view. These two separate views are slightly different and offset because the average person's eyes are about 2 inches apart. Therefore, each eye sees the world from a separate point in space and the brain puts them together to make a whole image.

To provide motion, or time-related simulation, the displayed images are updated frequently. This is similar to a movie projector where the individual displayed images provide the illusion of motion when the updating frequency is higher than about 24 Hz. Adding to the stereoscopic view, the simulator would need to double this frequency to update both the left and the right eye views.

The horizontal perspective display system promotes horizontal perspective projection viewing by providing the viewer with the means to adjust the displayed images to maximize the illusion viewing experience. By employing the computation power of the microprocessor and a real time display, the horizontal perspective display is capable of re-drawing the projected image to match the user's eyepoint with the camera eyepoint to ensure the minimum distortion in rendering the three dimension illusion from the horizontal perspective method. The system can further comprise an image enlargement/reduction input device, or an image rotation input device, or an image movement device to allow the viewer to adjust the view of the projection images. The input device can be operated manually or automatically.

The present invention simulator further includes various computer peripherals. Typical peripherals are space globe, space tracker, and character animation devices, which are having six degrees of freedom, meaning that their coordinate system enables them to interact at any given point in an (x, y, z) space.

With the peripherals linking to the simulator, the user can interact with the display model. The simulator can get the inputs from the user through the peripherals, and manipulate the desired action. With the peripherals properly matched with the physical space and the display space, the simulator can provide proper interaction and display. The peripheral tracking can be done through camera triangulation or through infrared tracking devices. Triangulation is a process employing trigonometry, sensors, and frequencies to “receive” data from simulations in order to determine their precise location in space.

The simulator can further include 3D audio devices. 3D audio also uses triangulation to send or project data in the form of sound to a specific location. By changing the amplitudes and phase angles of the sound waves reaching the user's left and right ears, the device can effectively emulate the position of the sound source. The sounds reaching the ears will need to be isolated to avoid interference. The isolation can be accomplished by the use of earphones or the like.

Similar to vision, hearing using one ear is called monoaural and hearing using two ears is called binaural. Hearing can provide the direction of the sound sources but with poorer resolution than vision, the identity and content of a sound source such as speech or music, and the nature of the environment via echoes, reverberation such as a normal room or an open field. Although we can hear with one ear, hearing with two ears is clearly better. Many of the sound cues are related to the binaural perception depending on both the relative loudness of sound and the relative time of arrival of sound at each ear. And thus the binaural performance is clear superior for the localization of single or multiple sound sources and for the formation of the room environment, for the separation of signals coming from multiple incoherent and coherent sound sources; and the enhancement of a chosen signal in a reverberant environment.

A 3D audio system should provide the ability for the listener to define a three-dimensional space, to position multiple sound sources and that listener in that 3D space, and to do it all in real-time, or interactively. Beside 3D audio system, other technologies such stereo extension and surround sound could offer some aspects of 3D positioning or interactivity. For better 3D audio system, audio technology needs to create a life-like listening experience by replicating the 3D audio cues that the ears hear in the real world for allowing non-interactive and interactive listening and positioning of sounds anywhere in the three-dimensional space surrounding a listener.

The head tracker function is also very important to provide perceptual room constancy to the listener. In other words, when the listener move their heads around, the signals would change so that the perceived auditory world maintain its spatial position. To this end, the simulation system needs to know the head position in order to be able to control the binaural impulse responses adequately. Head position sensors have therefore to be provided. The impression of being immersed is of particular relevance for applications in the context of virtual reality.

The eyes and ears often perceive an event at the same time. Seeing a door close, and hearing a shutting sound, are interpreted as one event if they happen synchronously. If we see a door shut without a sound, or we see a door shut in front of us, and hear a shutting sound to the left, we get alarmed and confused. In another scenario, we might hear a voice in front of us, and see a hallway with a comer; the combination of audio and visual cues allows us to figure out that a person might be standing around the comer. Together, synchronized 3D audio and 3D visual cues provide a very strong immersion experience. Both 3D audio and 3D graphics systems can be greatly enhanced by such synchronization.

The biofeedback eyewear system further comprises various biofeedback devices for user's inputs and outputs. A typical biofeedback device is a brain wave electrode measurement such as an electroencephalographic (EEG) system. The brain wave biofeedback system can be used to balance the left and the right side of the brain using binaural beat. The biofeedback device typically comprises an EEG system to measure the brain left and right electrical signals to determine the brain wave imbalance, and an audio generator to generate a binaural beat to compensate for the unbalanced EEG frequencies.

Other biofeedback devices are skin conductance, or galvanic skin response to measure the electrical conductance of the external skin, a temperature measurement of the body, hand and foot, a heart rate monitoring, and a muscle tension measurement.

An application of the biofeedback eyewear system having a brain wave electrode is a method to balance the brain left side and the brain right side by using binaural beat. The system comprises an electroencephalographic (EEG) system to measure the brain left and right electrical signals, an audio generator to generate a binaural beat to compensate for the unbalanced EEG frequencies. The method includes measuring the brain wave frequency spectrum of the individual, selecting the frequency exhibiting imbalanced behavior, and generating a binaural beat of that frequency.

The binaural beat can be generated by applying two different frequencies to two ears. The applied frequencies can range from 50 Hz to 400 Hz. The amplitudes and waveforms of the audio frequencies can vary to achieve best results for different users.

A computer is preferably used in the present invention for controlling the equipment. The binaural beat can be generated through electronic synthesizer or a frequency generator. The measurement of the brain wave is preferably by the use of an EEG equipment, but any other brain scan equipment can be used.

The method first measures the left and right brain wave frequencies of the individual by use of electroencephalographic (EEG) to determine the brain wave imbalance, then entraining the brain wave frequency of the individual at a chosen imbalanced brain wave frequency to improve the brain wave balance at that particular frequency. The system uses the EEG feedback to ensure of the proper balancing treatment.

One of the first “brain scan”, the EEG, or electroencephalograph, is still very useful in non-invasively observing the human brain activity. An EEG is a recording of electrical signals from the brain made by hooking up electrodes to the subject's scalp, typically placed on the head in the standard ten-twenty configuration. These electrodes pick up electric signals naturally produced by the brain and send them to galvanometers (ampere meter) that are in turn hooked up to pens, under which graph paper moves continuously. The pens trace the signals onto the graph paper. Modem EEG equipment now uses electronics, such as computer, to store the electric signals instead of using pens and graph papers.

EEGs allow researchers to follow electrical impulses across the surface of the brain and observe changes over split seconds of time. An EEG can show what state a person is in—asleep, awake, anaesthetized—because the characteristic patterns of current differ for each of these states. One important use of EEGs has been to show how long it takes the brain to process various stimuli.

The electrical activity, or EEG, of human brains has traditionally been used as a diagnostic marker for abnormal brain function and related symptomatic dysfunction. Often, traumatic disturbances such as mechanical injury, social stress, emotional stress and chemical exposure cause neurophysiological changes that will manifest as EEG abnormalities. However, disruption of this abnormal EEG activity by the application of external electrical energy, henceforth referred to as a neurostimulation signal, may cause yet further neurophysiological changes in traumatically disturbed brain tissues, as evidenced in an amelioration of the EEG activity, and hence are beneficial to an individual. Such therapeutic intervention has proven useful in pain therapy and in treating a number of non-painful neurological deficits such as depression, attention deficit disorder, and many others.

It is indicated that a beat frequency can be produced inside of the brain by supplying signals of different frequencies to the two ears of a person. The binaural beat phenomenon was discovered in 1839 by H. W. Dove, a German experimenter. Generally, this phenomenon works as follows. When an individual receives signals of two different frequencies, one signal to each ear, the individual's brain detects a phase difference or differences between these signals. When these signals are naturally occurring, the detected phased difference provides directional information to the higher centers of the brain. However, if these signals are provided through speakers or stereo earphones, the phase difference is detected as an anomaly. The resulting imposition of a consistent phase difference between the incoming signals causes the binaural beat in an amplitude modulated standing wave, within each superior olivary nucleus (sound processing center) of the brain. It is not possible to generate a binaural beat through an electronically mixed signal; rather, the action of both ears is required for detection of this beat.

Binaural beats are auditory brainstem responses which originate in the superior olivary nucleus of each hemisphere. They result from the interaction of two different auditory impulses, originating in opposite ears, below 1000 Hz and which differ in frequency between one and 30 Hz. For example, if a pure tone of 400 Hz is presented to the right ear and a pure tone of 410 Hz is presented simultaneously to the left ear, an amplitude modulated standing wave of 10 Hz, the difference between the two tones, is experienced as the two wave forms mesh in and out of phase within the superior olivary nuclei. This binaural beat is not heard in the ordinary sense of the word (the human range of hearing is from 20-20,000 Hz). It is perceived as an auditory beat and theoretically can be used to entrain specific neural rhythms through the frequency—following response (FFR)—the tendency for cortical potentials to entrain to or resonate at the frequency of an external stimulus. Thus, it is theoretically possible to utilize a specific binaural-beat frequency as a consciousness management technique to entrain a specific cortical rhythm.

When signals of two different frequencies are presented, one to each ear, the brain detects phase differences between these signals. Under natural circumstances a detected phase difference would provide directional information. The brain processes this anomalous information differently when these phase differences are heard with stereo headphones or speakers. A perceptual integration of the two signals takes place, producing the sensation of a third “beat” frequency. The difference between the signals waxes and wanes as the two different input frequencies mesh in and out of phase. As a result of these constantly increasing and decreasing differences, an amplitude-modulated standing wave—the binaural beat—is heard. The binaural beat is perceived as a fluctuating rhythm at the frequency of the difference between the two auditory inputs.

As a result, binaural beats are produced and are perceived by the brain as a result of the interaction of auditory signals within the brain. Such binaural beats are not produced outside of the brain as a result of the two audio signals of different frequencies. In a sense, the binaural beats are similar to beat frequency oscillations produced by a heterodyne effect, but occurring within the brain itself. However, the article discusses the use of such binaural beats in a strobe-type manner. In other words, if the brain is operating at one frequency, binaural beats of a fixed frequency are produced within the brain so as to entice the brain to change its frequency to that of the binaural beats and thereby change the brain state.

The binaural beat phenomenon described above also can create a frequency entrainment effect. If a binaural beat is within the range of brain wave frequencies, generally less than 30 cycles per second, the binaural beat will become an entrainment environment. This effect has been used to study states of consciousness, to improve therapeutic intervention techniques, and to enhance educational environments.

As the brain slows from beta to alpha to theta to delta, there is a corresponding increase in balance between the two hemispheres of the brain. This balanced brain state is called brain synchrony, or brain synchronization. Normally, the brain waves exhibit asymmetrical patterns with one hemisphere dominant over the other. However, the balanced brain state offers deep tranquility, flashes of creative insight, euphoria, intensely focus attention, and enhanced learning abilities. Thus it is important for the creative activity of the individual to have a “correct” balance and communication between the brain halves.

Deep relaxation technique combined with synchronized rhythms in the brain has been proven to provide the ability to learn over five times as much information with less study time per day, and with greater long term retention, and is credited to alpha wave production.

The left brain half is verbal, analytical and logical in its functioning, while the right is musical, emotional and spatially perceptive. The left brain hemisphere thinks in words and concepts, and the right thinks in pictures, feelings and perceptions. In a normal brain, a spontaneous shift in balance occurs between left and right, depending on what one is doing. When one is reading, writing and speaking, the left half will be more active than the right. On the other hand, when one is listening to music or is engaged in visual spatial perception, then the right half is most active.

By calculating the ratio between the amount of alpha waves in the right and left brain hemispheres, an expression for the balance between the brain halves is obtained, the so-called R/L ratio. If there is exactly the same amount of alpha waves in the right and left brain hemispheres, the RIL ratio will be 1.00. If there is more alpha in the right brain half, the R/L ratio will be more than 1.00, and vice versa, the RIL ratio will be less than 1.00 if there is more alpha in the left brain half.

In most people during rest with closed eyes, the R/L ratio is normally slightly above 1.00. This is probably due to our culture's emphasis on the functions of the left brain half. During deep relaxation, however, a balance of 1.00 between the brain halves is approached.

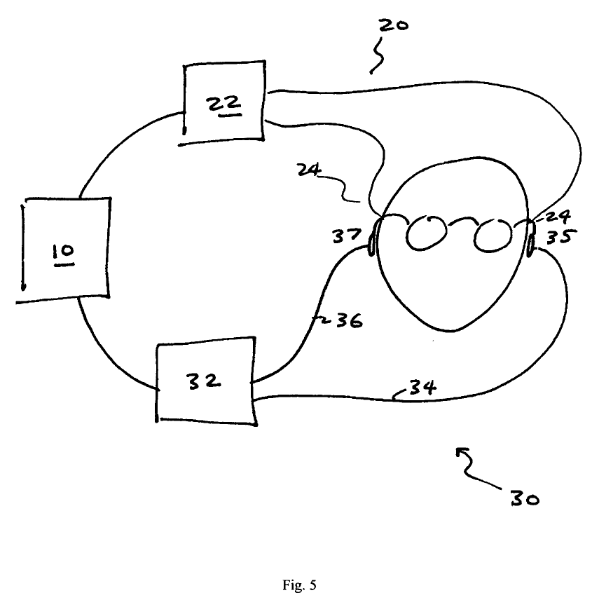

Shown in FIG. 5 is the present invention apparatus, comprising a computer 10 for controlling the equipment, an EEG system 20 to measure the brain wave spectrum, and a binaural beat system 30 to generate a binaural beat. The EEG system comprises an amplifier 22 and a plurality of electrodes 24 of the biofeedback eyewear system. The number of electrodes 24 is even and at least 2, one for each half of the brain, but can be as many as 4 or 6. The electrodes 24 and amplifier 22 can communicate with the computer 10. The binaural beat system 30 comprises a generator 32 to generate a first signal at a first frequency on a first channel 34 and a second signal at a second frequency on a second channel 36. The frequency difference between the first and second signals creates the binaural beat corresponding to a chosen imbalance brain wave frequency. First channel 34 send the first signal to one ear of the user through an earphone 35, and second channel 36 send the second signal to the other ear of the user through an earphone 37. These earphones are part of the biofeedback eyewear system. The binaural beat system 30 is responsive to the computer 10. There are optional devices such keypad, keyboard, mouse and display for conventional input and output devices, and volume, waveform, and balance controls for adjusting to the individual user and the purpose of the use.

In another embodiment of the invention, either or both the electrodes 24 and the earphones 35, 37 are wireless, and communicate with the amplifier 22 and the signal generator 32 wirelessly. The electrode 24 can be a modified eyewear handle, the cover part of the earphone, the outer part of the earphone, or the muffle of the earphone.

Generally, the binaural beat frequency that the brain can detect, ranges from approximately 0 to 100 Hz. The ear has the greatest sensitivity at around 1000 Hz. However, this frequency is not pleasant to listen to, and a frequency of 100 Hz is too low to provide a good modulation index. Thus the frequencies between 100 Hz and 1000 Hz are normally used for binaural beat, and preferably between 100 Hz and 400 Hz. Typically, the frequency of 200 Hz is a good compromise between sensitivity and pleasing sounds.

Thus according to the present invention, a constant frequency of 200 Hz audio signal can supplied to one ear (for example, the left ear) and another audio signal having a frequency which ranges from 300 Hz to 200 Hz is applied to the other ear (for example, the right ear). As a result, binaural beats at 0-100 Hz are produced in the brain. The audio signals can be toggled, meaning the constant frequency can be applied to the right ear and the varied frequency applied to the left ear. Further the toggle can happen at a fast rate. This toggle rate can help to maintain the attention span of the brain during the binaural beat generation and might allow the user to perceive the signal moving back and forth between the left and right ears. Further, the left and right ear signals can have different time delay or phase differences since, for low frequencies of this nature, the time delay or phase difference between the left and right signals could produce a greater effect than the relative amplitude to the brain. The time delay could be up to a few seconds and the phase difference can be anywhere from 0 to 360°.

The above audio signals can be produced in a plurality of ways. For example, an audio signal generator can be used to produce the audio signals and listened to through headphones. The audio signal can be computer generated. A computer program can be written to produce the required sound. Alternatively, analog operational amplifiers and other integrated circuitry can be provided in conjunction with a set of headphones to produce such audio signals. These signals may be recorded on a magnetic tape which the person listens to through a set of earphones. Headphones are necessary because otherwise the beat frequency would be produced in the air between the two speakers. This would produce audible beat notes, but would not produce the binaural beats within the brain.

The binaural beat can have various waveforms such as square, triangular, sinusoidal, or the various musical instruments. It is known that sound may be defined by its frequency, amplitude, and wave shape. For example, the musical note A has the frequency of 440 Hz, and the amplitude of that note is expressed as the loudness of the signal. However, the wave shape of that note is related strongly to the instrument used. An A played on a trumpet is quite different from an A played on a violin.

The present invention employs the EEG signals feedback to ensure proper application of the binaural beat. First, a brain frequency spectrum of a user is obtained through the EEG electrodes and EEG amplifier. From the spectrum, imbalanced frequencies are observed. The user then selects an imbalanced frequency to address. The brain frequencies are related to the human consciousness through various activities and enhancements such as better learning, better memory retention, better focus, better creativity, better insight, or just simply brain exercise, and thus instead of choosing a frequency, the user can just choose a desired enhancement. Then a binaural beat is applied using the selected frequency by audio inputs.

There is various brain balancing procedure. For example, the binaural beat can be continuous or intermittent. The binaural beat at the desired frequency can be maintained for some predetermined period of time, after which a new desired frequency can be determined. Another possibility would be to take the user to a rest frequency between sessions. Another possibility would be to allow the user to rest between sessions, e.g. generating no signal at all for a period of time. The amplitude and waveform of the applied frequencies can be constant, selected by the user, or vary. The binaural beat can start at the desired frequency, or can start at a higher or lower frequency and then moves toward the desired frequency. The binaural beat can phase lock onto a certain brain wave frequency of the person and to gently carry down to the desired frequency. The scanning or continuously varying frequency can be important since the different halves generally operate at different brain frequencies.

This is because one brain half is generally dominant over the other brain half. Therefore, by scanning at different frequencies from a higher frequency to a lower frequency, or vice versa, each brain half is locked onto the respective frequency and carried down or up so that both brain halves are operating synchronously with each other and are moved to the desired frequency brain wave pattern corresponding to the chosen state.

Synchronized brain waves have long been associated with meditative and hypnologic states, and audio with embedded binaural beats has the ability to induce and improve such states of consciousness. The reason for this is physiological. Each ear is “hardwired” to both hemispheres of the brain. Each hemisphere has its own olivary nucleus (sound-processing center) which receives signals from each ear. In keeping with this physiological structure, when a binaural beat is perceived there are actually two standing waves of equal amplitude and frequency present, one in each hemisphere. So, there are two separate standing waves entraining portions of each hemisphere to the same frequency. The binaural beats appear to contribute to the hemispheric synchronization evidenced in meditative and hypnologic states of consciousness. Brain function is also enhanced through the increase of cross-colossal communication between the left and right hemispheres of the brain.

How can audio binaural beats alter brain waves? We know that the electrical potentials of brain waves can be measured and easily quantified, such as EEG patterns. As to the second question raised in the above paragraph, audio with embedded binaural beats alters the electrochemical environment of the brain. This allows mind-consciousness to have different experiences. When the brain is entrained to lower frequencies and awareness is maintained, a unique state of consciousness emerges. This state is often referred to as hypnogogia “mind awake/body asleep.” Slightly higher-frequency entrainment can lead to hyper suggestive states of consciousness. Still higher-frequency EEG states are associated with alert and focused mental activity needed for the optimal performance of many tasks.

Synchronizing the left and right hemispheres allows the left brain to recognize the black and white words and smoothly transfer the meaning in color, motion, emotion etc. to the right brain to be converted into understandable thoughts that are easy to remember.

The present invention can affect various types of balancing brain activity.

In all of the embodiments which will be discussed hereinafter in more detail, it is essential that an audio signal be produced in which the frequency thereof or binaural beats produced thereby passes through the then operating brain-wave frequency of the person in order to lock onto and balance the brain-wave frequency. It is known that telling a stressed person to relax is rarely effective. Even when the person knows that he must try to relax, he usually cannot. Meditation and other relaxation methods seldom work with this type of person. Worrying about being stressed makes the person more stressed, producing a vicious cycle.

Another type is to raise the brain wave frequency, and particularly, to increase the performance of the person, for example, in sporting events. In this mode, both ears of the person are supplied with the same audio signal having a substantially continuously varying frequency which varies, for example, from 20 Hz to 40 Hz, although the signals are amplitude and/or phase modulated. It is believed that, if the brain wave frequency of the person is less than 20 Hz, the brain will phase lock onto audio signals of the same frequency or multiples of the same frequency. Thus, even if the brain is operating at a 10 Hz frequency rate, when an audio signal of 20 Hz is supplied, the brain will be phase locked onto such a signal and will be nudged up as the frequency is increased. Without such variation in frequency of the audio signal, the brain wave frequency will phase lock thereto, but will not be nudged up. Preferably, the audio signal changes from 20 Hz to 40 Hz in a time period of approximately 5 minutes and continuously repeats thereafter so as to nudge the brain frequency to a higher frequency during each cycle.

In view of the foregoing, it is one object of the invention to provide a method of inducing states of consciousness by generating stereo audio signals having specific wave shapes. These signals act as a carrier of a binaural beat. The resulting beat acts to entrain brain waves into unique waveforms characteristic of identified states of consciousness.

As will be discussed below, different regions of the brain produce distinct electrical waveforms during various physical, mental, and emotional states of consciousness. In the method of the invention, binaural beat audio wave shapes are made to match such particular brain waves as they occur during any mental physical, and emotional human condition of consciousness. Thus, it is possible to convert waveforms from specific brain regions, as well as complete brain surface electrical topography.

Many times the brain wave patterned is locked, and thus a disruption of the locked brain is necessary to bring the brain back to the synchronizing state, and to re-establish the biological systems flexibility. The present method uses the EEG measurements to identify regions of the brain that need work, and the binaural beat technique to exercise the brain. The locations of the EEG electrodes can be anywhere near the center of the forehead which are near the dominant brain wave frequency.

The EEG measures the brain wave with different frequencies to establish the frequency spectrum. The frequency spectrum might also be obtained from a transformation of the brain wave frequency measurements. Such a transform may include, but not be limited to, a compression, expansion, phase difference, statistical sampling or time delay from the brain wave frequency.

It is preferred that the working time be between one second and one hour. It is more preferred that the time be between 1 and 30 minutes. It is even more preferred that the time is between 1 minute and 10 minutes.

What is claimed is:

1. A method of biofeedback employing a biofeedback eyewear system, the biofeedback eyewear system comprising
a mutually exclusive eyeglasses for stereoscopic viewing; and
a plurality of electrodes for measuring biological data of the wearer, the electrodes being incorporated in a component of the eyewear that contacts the wearer;
the method comprising the steps of
providing stereoscopic horizontal perspective viewing through the mutually exclusive eyeglasses;
providing biological measurements through the electrodes; and
displaying the biological measurements to the horizontal perspective viewing.

2. A method as in claim 1 wherein the biological measurements comprise the measuring of brain wave activities.

3. A method as in claim 1 wherein the biological measurements comprise the measuring of skin conductance.

4. A method as in claim 1 wherein the biological measurements comprise the measuring of body temperature.

5. A method as in claim 1 wherein the biological measurements comprise the measuring of heart rate.

6. A method as in claim 1 wherein the biological measurements comprise the measuring of muscle tension.

7. A method as in claim 1 wherein the mutually exclusive eyeglasses employ a method of anaglyph, polarized glasses, shuttering glass, optical lenses or lenticular lenses.

8. A method as in claim 1 wherein the mutually exclusive eyeglasses comprises a linear polarized lenses having 90° polarization.

9. A method as in claim 1 wherein the component of the eyewear is the handle, the frame, or the bridge of the eyewear.

10. A method as in claim 1 further comprising a step of tracking for eyepoint or earpoint.

11. A method of biofeedback employing a biofeedback eyewear system, the biofeedback eyewear system comprising
a stereo earphone for binaural hearing; and
a plurality of electrodes for measuring biological data of the wearer, the electrodes being incorporated in a component of the eyewear that contacts the wearer;
the method comprising the steps of
providing binaural hearing throuh the stereo earphone;
providing biological measurements through the electrodes; and
providing biofeedback by binaural hearing corresponding to the biological measurements.

12. A method as in claim 11 wherein the biological measurements comprise the measuring of brain wave activities, the measuring of skin conductance, the measuring of body temperature, the measuring of heart rate, or the measuring of muscle tension.

13. A method as in claim 11 wherein the component of the eyewear is the handle, the frame, or the bridge of the eyewear.

14. A method as in claim 11 further comprising a step of tracking for eyepoint or earpoint.

15. A method of biofeedback employing a biofeedback eyewear system, the biofeedback eyewear system comprising
a mutually exclusive eyeglasses for stereoscopic viewing;
a stereo earphone for binaural hearing; and
a plurality of electrodes for measuring biological data of the wearer, the electrodes being incorporated in a component of the eyewear that contacts the wearer;
the method comprising the steps of
providing stereoscopic horizontal perspective viewing through. the mutually exclusive eyeglasses;
providing binaural hearing throuh the stereo earphone;
providing biological measurements through the electrodes;
providing biofeedback by binaural hearing corresponding to the biological measurements; and
displaying the biofeedback to the horizontal perspective viewing.

16. A method as in claim 15 wherein the biological measurements comprise the measuring of brain wave activities, the measuring of skin conductance, the measuring of body temperature, the measuring of heart rate, or the measuring of muscle tension.

17. A method as in claim 15 wherein the mutually exclusive eyeglasses employ a method of anaglyph, polarized glasses, shuttering glass, optical lenses or lenticular lenses.

18. A method as in claim 15 wherein the mutually exclusive eyeglasses comprises a linear polarized lenses having 90° polarization.

19. A method as in claim 15 wherein the component of the eyewear is the handle, the frame, or the bridge of the eyewear.

20. A method as in claim 15 further comprising a step of tracking for eyepoint or earpoint.
